(19) United States
(12) Patent Application Publication
(10) Pub. No.: US 2022/0176005 A1
(43) Pub. Date: Jun. 9, 2022

(54) SYSTEM AND METHOD FOR UVC AIRSTREAM DISINFECTION

(71) Applicant: James G. Riesenberger, Canton, GA (US)

(72) Inventor: James G. Riesenberger, Canton, GA (US)

(21) Appl. No.: 17/543,142

(22) Filed: Dec. 6, 2021

Related U.S. Application Data
(60) Provisional application No. 63/121,347, filed on Dec. 4, 2020.

(51) Int. Cl. A61L 9/20

(52) U.S. Cl. CPC A61L 9/20; A61L 2209/15; A61L 2209/12

(57) ABSTRACT

A system and method for airstream disinfection of vehicle heating, ventilation, and air conditioning disinfection. Modifications to existing systems are disclosed which provide the housing and electrification for UVC bulbs within vehicle air handling systems in order to provide adequate disinfection of moving air during occupancy of an enclosed-space vehicle during operation without exposing the drivers and passengers to harmful UVC radiation. Disinfection with UVC radiation is known to accomplish the killing and/or neutralization of microorganisms suspended within air, thereby allowing vehicle occupants to breath air which is constantly disinfected. Components of the disclosed system include UVC lights, a converter, ballasts, interconnecting wiring, a relay, electrical connections, and a modified system enclosure. A method for installing these components to provide the intended effect of disinfection of vehicle airstreams includes modification and/or installation of an airstream enclosure to house UVC bulbs, installation and powering of the bulbs.

### CROSS-REFERENCE TO RELATED APPLICATIONS

To the full extent permitted by law, the present United States Non-Provisional Patent Application hereby claims priority to and the full benefit of, United States Provisional Application entitled “UVC AIRSTREAM DISINFECTION FOR MACK [GRANITE] TRUCK,” having assigned Ser. No. 63/121,347, filed on Dec. 4, 2020, which is incorporated herein by reference in its entirety.

### FEDERALLY SPONSORED RESEARCH OR DEVELOPMENT

None

### PARTIES TO A JOINT RESEARCH AGREEMENT

None

### REFERENCE TO A SEQUENCE LISTING

None

### BACKGROUND OF THE DISCLOSURE

### Technical Field of the Disclosure

The instant disclosure relates to vehicles, namely passenger and commercial vehicle interior air handling systems. More particularly, the instant disclosure relates to the manufacture and installation of an improved air handling component to introduce disinfecting UVC light therein.

### Description of the Related Art

Improvements in commercial and passenger motor vehicle air handling designs have improved overall vehicle air quality, primarily through implementation of air filtration technology. Nevertheless, new vehicles, including passenger cars, trucks, vans, etc. as well as buses, tractor trailers, ambulances, etc. may fail to filter airborne microscopic viral and bacterial organisms due to particle versus filter pore size. Additionally, filtering systems having very small pores sufficient to suspend airborne organisms may significantly impact overall airflow through a vehicle airstream system. This often means a trade-off between volume/rate of airflow and filtration ability which can inevitably cause inefficiencies in an airstream system. Installation of even very high-filtration systems thus decreases the overall rate at which air may pass through a system, which may then be compensated for by running a system at a higher fan speed. Nevertheless, even through use of very fine filters, small organisms and/or the droplets in which they may be suspended, may still pass through such a filter and remain suspended within a vehicle compartment until it is either ingested (breathed or otherwise deposited on human tissue), deposited on a surface, exited via ventilation, or caught in a second pass through the filter. The ideal or the best indoor air quality filtration systems, such as those found in state-of-the-art hospitals, usually house HEPA filters which may be rated at 0.3-1 microns. Minimum Efficient Reporting Value (MERV) may be used to quantify and/or rate this tradeoff between filtering power and efficiency. Filters having high MERV rating and small particulate-size capturing capabilities, usually eliminate, or filter out over 99% of airborne particulates smaller than this size, which should capture many airborne droplets greater than this size. However, if the organisms themselves are smaller or dry out and become part of a smaller airborne particle, it may pass through such filtering media.

In response to the recent emergence of SARS-CoV-2 as an infectious cause of COVID-19, many infectious disease experts have developed measures to contain and/or limit its spread among individuals as they interact indoors with one another. These have involved widespread adoption of universal masking policies indoors, vaccination campaigns and/or mandates, surveillance testing for the virus's presence, social/physical distancing measures, and other restrictions and/or counter measures seeking to limit the spread of SARS-CoV-2 among the global population. Each has achieved some level of spread-reduction, though the global pandemic continues to affect individuals' daily lives through such restrictions and attempts to contain the virus. Air quality experts in particular have offered multiple solutions, both previously-known and novel, in order to limit the spread of the virus indoors. These include but are not limited to increasing the ventilation of indoor spaces (i.e., bringing in air from outside), ionization, ozone treatment and UVC air treatment. Each implementation brings with it various tradeoffs, much like the restrictions and prevention measures adopted and/or imposed on individuals. Increasing ventilation in climate-controlled spaces by definition causes inefficiencies due to the need to heat and/or cool outside air to maintain a comfortable indoor temperature. Since outdoor air is generally thought to be free of viral infectious organisms, this tradeoff has been readily adapted despite the related inefficiencies. However, other tradeoffs may exist with this strategy including environmental air contamination (especially in urban areas), allergen introduction which may require additional filtration, and heating, ventilation, and air-conditioning (HVAC) system wear. UVC (ultraviolet radiation) air treatment has been readily adopted by schools, hospitals, businesses, and other building types as a way to inactivate/eliminate pathogens in the air non-specifically. Once a pathogen is inactivated, it cannot reproduce and the air may be considered disinfected.

All radiation is a form of energy, most of which is invisible to the human eye. UV radiation is only one form of radiation and it is measured on a scientific scale called the electromagnetic (EM) spectrum. UV radiation is a type of EM energy along a specific band of the EM spectrum. Radio waves are EM waves which transmit sound from a radio station's tower to, for instance a stereo, or between cell phones. Microwaves, like those that heat food in a microwave oven are another type of EM radiation as well as visible light that is emitted from the lights in a home. UV radiation, specifically, is the portion of the EM spectrum between X-rays and visible light. This UV spectrum is broken into at least three types: UVA, UVB, and UVC. UVA rays have the longest wavelength, followed by UVB and UVC, which has the shortest. All three are emitted by Earth's Sun, but only UVA and UVB are thought to reach Earth's surface. UVC radiation from the sun does not reach Earth's surface because it is blocked by the ozone layer of the Earth's atmosphere. Thus, the only way humans can be exposed to UVC radiation (at least while on Earth), is through an artificial source, such as a lamp or laser having UVC-emission properties. Like all UV radiation, UVC radiation can cause mild and even severe burns of the skin and eye injuries (photokeratitis). Individuals are advised to avoid direct skin exposure to UVC radiation and never look directly into a UVC light source, even briefly. Skin burns and eye injuries from UVC exposure usually resolve within a week with no known long-term damage. Due to the short wavelength of UVC, the penetration depth of UVC radiation is very low, and the risk of skin cancer, cataracts or permanent vision loss is also thought to be very low. The type of eye injury associated with exposure to UVC causes severe pain and a feeling of having sand in the eyes. Sometimes people are unable to use their eyes for one to two days. It can occur after a very short exposure (seconds to minutes) to UVC radiation.

UVC radiation, specifically, is a known disinfectant for air, water, and nonporous surfaces. UVC radiation has effectively been used for decades to reduce the spread of bacteria, such as tuberculosis. For this reason, UVC lamps are often called “germicidal” lamps. Prior to the emergence of SARS-CoV-2, hospitals may have been the primary adoptees of this technology with respect to air handling systems. This could prevent infection in surgical settings, protect immunocompromised individuals, and limit transmission of infectious agents among patients, staff, and visitors in hospitals. Due to the concerns regarding human safety, utilization of UVC treatment may involve installation and/or use in an unoccupied where a UVC bulb may illuminate the space with UVC radiation during periods the room is unoccupied (e.g., an office at night) or installation within an HVAC system, which would not be occupied by individuals within any particular building and can be used to treat air as it passes out of occupied space and into the system. In the case of the former, treatment of occupied space, the installation may be permanent with occupancy sensors or may be temporary and portable, which may be rolled into a room during a period of unoccupancy. During maintenance, installation, and other activities requiring individuals to access the UVC lamps, they may require protective equipment and/or a simple switch to deactivate during a potential exposure.

Specific to SARS-CoV-2, UVC radiation has been shown to destroy the outer protein coating of other individual SARS-Coronavirus organisms, which may be different virus from the current SARS-CoV-2 virus. Other studies show UVC's ability to create one or more dimers along the DNA or RNA strands of viruses, or otherwise affect their genetic material, causing the virus's replication capabilities to be interfered with, thereby stopping transmission. Many promising studies, investigations, and installations have yielded indication that this specific virus is no different than previously studied coronaviruses in this regard. The destruction of the outer protein coating and/or dimerization (or other radiation-caused damage) of its genetic material ultimately leads to inactivation of the virus. When viewed under a microscope, observers often remark that the organisms look like they have been “cooked” or “fried”. However, currently there is limited published data about the wavelength, dose, and duration of UVC radiation required to inactivate the SARS-CoV-2 virus. It is thought and/or hypothesized that other mechanisms of interaction between the viral organism(s) and UVC radiation may similarly kill, disinfect, and/or neutralize the virus in question. These include but are not limited to effects on viral DNA/RNA, other viral proteins (i.e., non-coating proteins such as the S1 spike protein), and/or other important viral cellular features which impact their virality and/or infectiousness.

In the past, vehicle airstream and/or vehicle HVAC systems were uncommonly candidates for installation of UVC or other air disinfection systems. Though reasons may vary, this may have been due to their expense, size, as well as the fact that most vehicles have limited occupancy. Due to the emergence of SARS-CoV-2 as a global infectious agent which has caused a pandemic, vehicle air quality has received renewed interest. Where previously, air systems which relied on heavy filtration of outside air in order to prevent introduction of pollutants and/or allergens into the vehicle cabin, interest has grown into the direction if increasing the ventilation and/or filtering recirculated air (rather than outdoor air being filtered upon introduction into the vehicle). While filtration of incoming air may still be important to vehicle occupant health, vehicle occupant safety now deserves a new focus—the disinfection of recirculated air, particularly during winter and summer operation. The challenges of fitting lamps capable of sufficiently “dosing” possibly contaminated air with UVC, as well as other challenges related to retrofitting existing vehicle HVAC systems with such lamps has remained a challenge. No known solution offers a ready-made solution for individual vehicle types which may be efficiently and economically run, though the need to protect certain individuals from being infected remains. In particular, drivers of vehicles which are involved in the transport of goods and individuals, as well as the passengers thereof, are highly critical to the maintenance of modern commerce and transport. Protection of those individuals from infection may be key to sustaining our modern economy, supply chain, and lifestyle.

Therefore, it is readily apparent that there is a recognized unmet need for a system and method for disinfecting vehicle air through use of UVC lamps. The instant disclosure is designed to address this need through installation of an apparatus within a vehicle HVAC system and methods of installation and use, which includes the systems, apparatuses, and methods disclosed herein while addressing at least some of the aspects of the problems discussed above.

### SUMMARY

Briefly described, in a possibly preferred embodiment, the present disclosure overcomes the above-mentioned disadvantages and meets the recognized need for such an UVC vehicle airstream/HVAC disinfection system and method of installation and use. The primary apparatus may involve one or more UVC lamps installed within one or more air-handling voids of a vehicle's HVAC system. Additional modifications may be required, depending on the size and configuration of specific vehicles. For instance, compartments/voids within a system through which air flows may require additional size to accommodate sufficiently powerful UVC lamps. Additionally, increased supporting structure may be required to both house the lamps and stabilize the compartment/void housing the lamps. Yet another modification may be supply of power to the UVC lamps sufficient to operate. Many other modifications may be required, which may involve the conversion of power, safety (from radiation and electrical shock), ease of installation, ease of maintenance, and other bulb protection/stability considerations.

More specifically, highly specific example embodiments of the present system and method are disclosed herein. Namely, the modification and installation of new air handling apparatuses and/or components, new power supplies/sources, new ballasts, new mounts/securing apparatuses, new wiring harnesses, new converters or inverters, and new enclosures may be required for UVC installations in commercial trucking vehicles. More specifically, these new modifications/installations may include those related to trucks manufactured, produced, sold, and/or serviced by VOLVO® Trucks North America, Mack® Trucks, Inc., and other truck manufacturing companies that manufacture various commercial trucking vehicles which can be observed travelling throughout the United States of America, and other countries worldwide. While modification of existing vehicles in commercial fleets may be a subject of this disclosure, original equipment may be modified at the time of initial manufacture to include the disclosed components, thereby making these features and components original equipment manufacturer (OEM) parts. Additionally, these features and components may be offered as standard equipment, optional OEM equipment, or dealer-installed and/or aftermarket equipment, as may be understood by those having ordinary skill in the art.

The devices, apparatuses, systems, and methods of the disclosure may provide various features and benefits, as will be understood by those having ordinary skill in the art. These include but are not limited to disinfection of vehicle interior airstreams at a specified airflow rate, simple/inexpensive/quick installation/maintenance/service, low-power consumption, limited vehicle performance reduction, limited and/or non-existent airflow reduction, sufficient UVC exposure time, multiple cabin air turnovers per hour, the like and/or combinations thereof.

In a possibly preferred exemplary embodiment, the devices, apparatuses, systems, and methods disclosed herein may relate specifically to Mack® Truck, Inc. vehicles manufactured from 2007 to present, as well as other vehicle systems manufactured by other manufacturers which may share some or many features with such vehicles. Additionally, the modifications, devices, apparatuses, and systems of the disclosure may specifically include but are not limited to UVC lamp(s), transformers, converters, inverters, ballast(s), interconnecting wiring, wiring harness(es) (e.g., Molex® wire-to-wire connectors), and air handling enclosures and the modification thereof. Additional modifications and installations may include holes/apertures and support brackets to accommodate the other modifications and installations.

These and other features of the disclosed airstream disinfection improvement and methods of installation and operation will become more apparent to one skilled in the art from the prior Summary and following Brief Description of the Drawings, Detailed Description of exemplary embodiments thereof, and Claims when read in light of the accompanying Drawings or Figures.

### BRIEF DESCRIPTION OF THE DRAWINGS

The disclosed apparatus and methods of use will be better understood by reading the Detailed Description with reference to the accompanying drawings, which are not necessarily drawn to scale, and in which like reference numerals denote similar structure and refer to like elements throughout, and in which:

FIG. 1 is a perspective cutaway view block diagram of an exemplary modified clamshell enclosure of the disclosure, the clamshell enclosure having disinfecting lamps installed therein.

FIG. 2 is a perspective view block diagram of the exemplary modified clamshell enclosure having its lamps ready for installation.

FIG. 3 is a perspective view block diagram of an exemplary electrical enclosure of the disclosure, the front of the enclosure is cutaway to show the internal components of the enclosure.

FIG. 4 is a perspective view block diagram of an exemplary system of the disclosure.

FIG. 5 is a perspective view of an unmodified clamshell enclosure.

FIG. 6 is a perspective view of an exemplary modified clamshell enclosure.

FIG. 7 is a flow diagram of an exemplary method of installation.

It is to be noted that the drawings presented are intended solely for the purpose of illustration and that they are, therefore, neither desired nor intended to limit the disclosure to any or all of the exact details of construction shown, except insofar as they may be deemed essential to the claimed disclosure.

### DETAILED DESCRIPTION

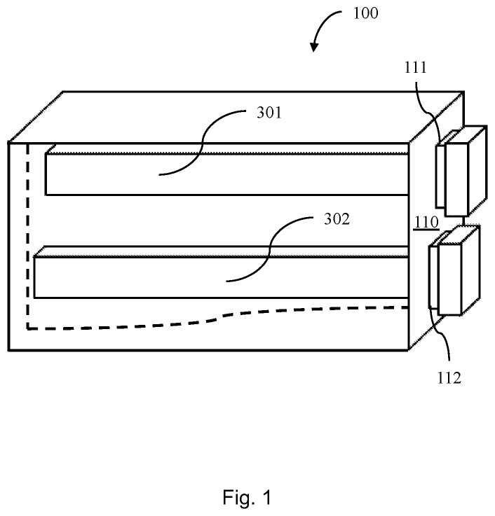

In describing the exemplary embodiments of the present disclosure, as illustrated in FIGS. 1-7, specific terminology is employed for the sake of clarity. The present disclosure, however, is not intended to be limited to the specific terminology so selected, and it is to be understood that each specific element includes all technical equivalents that operate in a similar manner to accomplish similar functions. Embodiments of the claims may, however, be embodied in many different forms and should not be construed to be limited to the embodiments set forth herein. The examples set forth herein are non-limiting examples, and are merely examples among other possible examples. It should be noted that the term vehicle and/or automobile may be used herein as a descriptor for any motorized means of transportation. The term disinfection as used herein shall mean any reduction in microorganism population within air through any means. Microorganisms shall mean any multi- or unicellular organism of small size and generally capable of replication within a larger lifeform to cause an infection thereof. The terms UV, UVC, and radiation, the like, and combinations thereof may be used to refer to any illumination, though invisible, which may introduce electromagnetic radiation into air. The terms airstream, HVAC, and vehicle air handling system may be used interchangeably to mean systems which are designed to move air within an indoor and/or vehicle space. Additionally, various components and/or steps of the disclosed system, apparatuses, devices, and methods may be described in various ways. By way of example and not limitation, a void through which air passes through such a system may be referred to as a compartment, enclosure, light reactor, clamshell enclosure, airstream, or simply void. Power, electricity, and other terms which refer specifically to the transmission of electrons to power electrical devices may be used herein to describe various modifications and installations of the disclosed system, apparatuses, devices, and the methods of installation, operation, use, and maintenance thereof.

Referring now to FIGS. 1-4 by way of example, and not limitation, therein are illustrated several perspective view block diagrams, some of which show cutaway portions, showing exemplary configurations of modifications to exemplary vehicle airstream systems and exemplary electrical components and lamps. Though some components may be illustrated therein to have a shape, size, and spatial relationship, the disclosure is not so limited. Illustrative and artistic choices therein are used solely for recognition purposes, including symbols and icons which may represent features and/or components of a vehicles which may or may not be a feature of the system of the disclosure.

Referring specifically to FIG. 1, therein illustrated is a perspective cutaway view block diagram of an exemplary airstream enclosure 100 of the disclosure, airstream enclosure 100 having disinfecting lamps 301, 302 installed therein. Generally speaking, vehicles having heating, ventilation, and air conditioning systems equipped therein feature an enclosure having a void or space through which air is pumped, usually via a fan and ducting, and then exits through various other ducts to distribute the air throughout the cabin. Airstream enclosure 100 may be thought of as an HVAC manifold, having properties of distributing positive-pressure air throughout a series of ducts. Due to airstream enclosure 100 general and universal adoption among vehicle designers and manufacturers, such an enclosure makes an excellent space for centrally disinfecting air as it is taken from one or more cabin intake location(s) and distributed to various outlets within the cabin of the vehicle. Therefore, by adding additional openings, ports, or apertures, such as first airstream enclosure aperture 111 and second airstream aperture 112 on airstream enclosure aperture side 110 may be capable of receiving lamps. Having made such a modification to airstream enclosure 100, first UVC bulb 301 and second UVC bulb 302 may protrude through first airstream enclosure aperture 111 and second airstream aperture 112, respectively, and into the void or space within airstream enclosure 100, such that it is airstream enclosure 100 itself which protects occupants from harmful UVC radiation. It should be noted that various solid objects, usually those that are tinted or opaque, are known to block UVC radiation, as well as blocking visible light by virtue of their tint and/or opacity. Suitable materials vary in OEM equipment, though opaque plastics are generally incorporated for airstream enclosure 100 in many vehicles. Though not drawn to scale, various important features may exist for first UVC bulb 301 and second UVC bulb 302. These may include, but are not limited to size and weight of bulb, durability of bulb, power supply requirements, light output, light intensity, expected bulb life, power consumption, the like and/or combinations thereof. Additionally, it should be noted that first UVC bulb 301 and second UVC bulb 302 may be bulbs having cap bases and/or sockets, which may require suitable receptacles be also installed, as may be understood by those of ordinary skill in the art, after a review of the remaining Detailed Description and accompanying Drawings. Additionally relevant to FIG. 1 may be a need to protect against long-term exposure to UVC radiation of the various components of airstream enclosure 100. UVC radiation may have deleterious effects upon many plastics. Steps such as incorporation of UVC-resistant or—stable plastics, treatment with a UVC-blocking or—tinting paint or coating, or application of a reflective material to the internally-exposed materials may each help prolong the useful life of airstream enclosure 100.

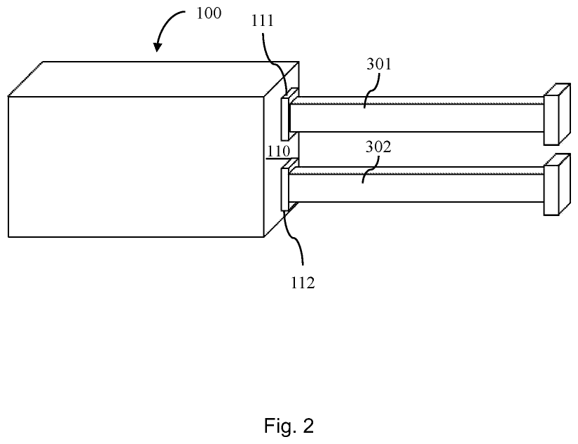

Referring specifically to FIG. 2, therein illustrated is a perspective view block diagram of airstream enclosure 100 having its first UVC bulb 301 and second UVC bulb 302 ready for installation. As detailed above, vehicles having HVAC systems equipped therein may generally feature an enclosure having a void or space through which air is pumped and then exits through various ducts to distribute the air throughout the cabin, each duct of which may have a controlled/controllable flow rate similar to a manifold. Due to airstream enclosure 100 general and near-universal adoption, such an enclosure may make an excellent space for centrally disinfecting air as it is taken from one or more intakes (or all intakes), heated/cooled and/or disinfected and directed toward various outlets within the cabin of the vehicle. Therefore, by adding additional openings, ports, or apertures, such as first airstream enclosure aperture 111 and second airstream aperture 112 on airstream enclosure aperture side 110, such a system of the disclosure may be capable of receiving first UVC bulb 301 and second UVC bulb 302, respectively. Having made such a modification to airstream enclosure 100, first UVC bulb 301 and second UVC bulb 302 may protrude through first airstream enclosure aperture 111 and second airstream aperture 112, respectively, and into the void or space within airstream enclosure 100, such that it is airstream enclosure 100 itself which protects occupants from harmful UVC radiation. Importantly, FIG. 2 may also illustrate the removal, replacement, and/or maintenance of first UVC bulb 301 and second UVC bulb 302. By using particularly long bulbs having a socket on one end, this replacement/repair/maintenance more convenient and less labor intensive. As may be readily apparent from a detailed review of FIGS. 1-2, additional stabilization for first UVC bulb 301 and second UVC bulb 302 may be installed in/on airstream enclosure 100 in order to operably combine with first UVC bulb 301 and second UVC bulb 302 (or brackets, sockets, etc. already operably combined with first UVC bulb 301 and second UVC bulb 302) so as to increase stability. Such a protrusion may be represented at/near airstream enclosure aperture 111 and second airstream aperture 112, as may be illustrated in FIGS. 1-2. Additionally, in addition to cap bases, it should be noted that first UVC bulb 301 and second UVC bulb 302 may require sufficient power and wiring to accompany suitable receptacles/sockets which may also be installed, as may be understood by those of ordinary skill in the art after a review of the remaining Detailed Description and accompanying Drawings.

Having installed such a system into a Mack® Truck, Inc. (VOLVO®) manufactured vehicle, one such of many suitable sizes for UVC bulb 301 and second UVC bulb 302 may include but are not limited to cap-base 2G11 having 4 pins, color code TUV (UVC), 0.85 A current, 225 mm in length, 35 W power rating, 254 nm spectrum, 11 W total UVC output, and an expected 9,000-hour useful life. Benefits of such a bulb configuration for first and second UVC bulbs 301, 302, the modified airstream system may further benefit from short-wave UV radiation with a peak at 253.7 nm (UVC) for disinfection purposes, a protective inside coating which may ensure constant UV output over the complete lifetime of first UVC bulb 301 and second UVC bulb 302, special lamp glass in order to filter out 185 nm ozone-forming radiation, high output first UVC bulb 301 and second UVC bulb 302 which may feature wind-chill correction for improved performance in moving air (thereby reducing amount of required lamps), and optimum UVC output per lamp length, allowing for reduction of system size. Additionally, warning signs on first UVC bulb 301 and second UVC bulb 302 may indicate that the lamp radiates UVC. Such warning may also exist on airstream enclosure 100, subsequent to modification. More generally speaking, long lamps having UVC germicidal properties may be the best mode of application, assuming the length may be accommodated with minimal or no modification to the physical size of airstream enclosure 100, such that the overall dimensions of airstream enclosure 100 may change to a small or even non-existent degree so as to ensure compatibility in vehicle areas which may be exceedingly tightly organized.

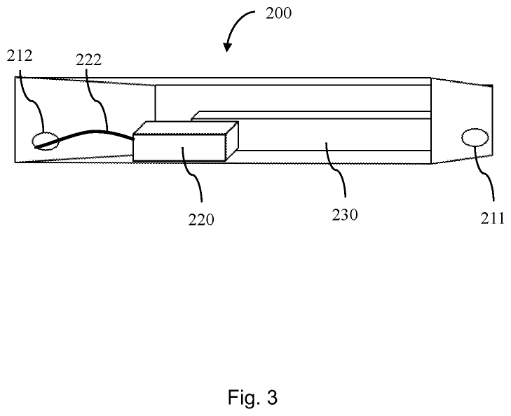

Referring specifically to FIG. 3, therein illustrated is a perspective view block diagram of an exemplary electrical enclosure 200 of the disclosure, the front of the enclosure is cutaway to show an exemplary arrangement of internal components of exemplary electrical enclosure 200. Electrical enclosure 200 may include one or more openings, such as electrical enclosure first opening 211 and electrical enclosure second opening 212. Openings may be useful for installation and/or protrusion of wring capable of electrifying first UVC bulb 301 and second UVC bulb 302. As may be understood by those ordinary having skill in the art of vehicle manufacture may appreciate, electrical enclosure 200 may be installed in close proximity of airstream enclosure 100, but may also be installed remotely and operably combine with first UVC bulb 301 and second UVC bulb 302 and a power source through wiring. Such wiring may be 18 gauge, or larger or smaller, depending on distance needed and other electrical considerations. Additionally, fuses, potentiometers, switches, relay devices, the like and/or combinations thereof may be installed therebetween electrical enclosure 200 and first UVC bulb 301 and second UVC bulb 302. Turing toward the components of electrical enclosure 200, therein may reside ballast 230 which may be operably connected to power converter 220 and terminate through electrical enclosure second opening 212 via power source wiring 221, which may also feature various fuses, potentiometers, switches relay devices, the like and/or combinations thereof.

Having installed such a system into a Mack® Truck, Inc. (VOLVO®) manufactured vehicle, one such of many potentially suitable ballast 230 and power converter 220 are herein disclosed, by way of example and not limitation. Ballast 230 may feature a normal input voltage of 24V, with an input current of 2.5 A(24V), an input current total harmonic distortion of less than 10%, an operating frequency of >35 kHz, a lamp current crest factor of 1.7 or less and a UL listed Class “P” Indoor rating (Type 1). Ballast 230 may also feature a programmed start feature to ensure UVC bulbs 301 and 302 maintain optimal life. Such a ballast may ensure efficiency, constant current regulation, and thereby longevity of first UVC bulb 301 and second UVC bulb 302. Power converter, in this potentially preferred embodiment, may feature 12-24 VDC (10 A-240 W) so as to properly transform/convert power from a 12V DC source to first UVC bulb 301 and second UVC bulb 302 as herein described.

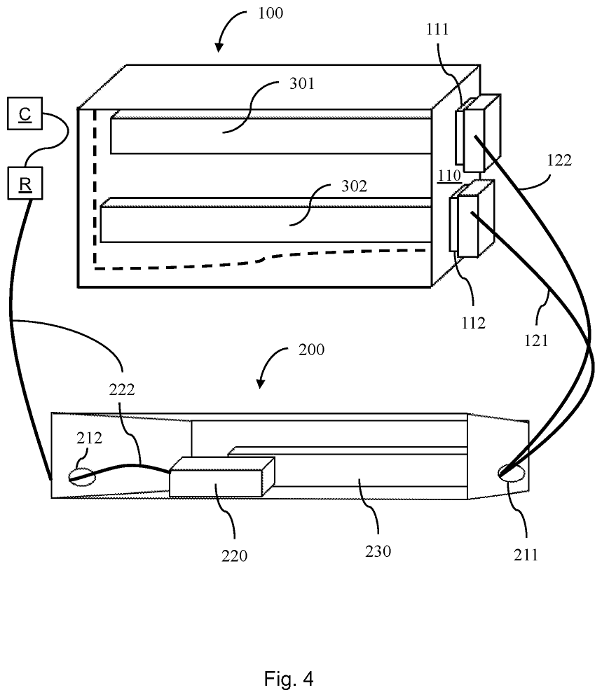

Referring specifically to FIG. 4, therein illustrated is a perspective view block diagram of an exemplary system of the disclosure, including airstream enclosure 100, electrical enclosure 200, and the corresponding features and interconnections thereof. FIG. 4 generally includes the features and components illustrated and described therein FIGS. 1-3. Starting at what may be a best power source for a system of the disclosure, circuit C, which may be specifically responsible for powering one or more fans of an HVAC system, may generally supply power to the system so long as sufficient power to fire and run UVC bulbs 301 and 302 may be obtained from circuit C of a vehicle fan accessory circuit C. Circuit C may contribute a 12V DC power source and thereby power features of the disclosed system directly when circuit C of the fan is in the “on” position, or which may be from another sufficient circuit C, which may then be relayed via permissive relay R to power converter 220, which may be capable of converting DC power to AC power required for powering first UVC bulb 301 and second UVC bulb 302 via ballast 230. Another alternative exists where power may be obtained from another circuit C source but requires the incorporation of a “permissive” relay, which is powered only when the fan accessory is switched to the “on” position within the operator cabin. When this relay is energized, it closes contacts to the circuit powering the lamps (i.e., UVC bulbs 301 and 302) within the light reactor (i.e., airstream enclosure 100). The incorporation of relay R, prohibits energization of UVC bulbs 301 and 302 when the fan is switched to the “off” position. Yet another option may be to incorporate the use of an inverter [thereby eliminating the converter] which is powered by one of the vehicle's 12V DC circuits and changes the power to 120V AC (alternating current). This current may then be fed directly to the ballasts accepting 120V AC current and then in turn to the lamps. For avoidance of doubt, circuit C of the system illustrated in FIG. 4 may be any circuit capable of powering a system as herein described and relay R may simply be configured to signal that an HVAC fan is on such that UVC bulbs 301 and 302 may safely operate or simply switch on and off, depending on the on/off state of circuit C of an accessory HVAC fan. As described above, first UVC bulb 301 and second UVC bulb 302 are contained within airstream enclosure 100, and may be therein mounted and/or secured and powered via the systems as illustrated therein FIG. 4.

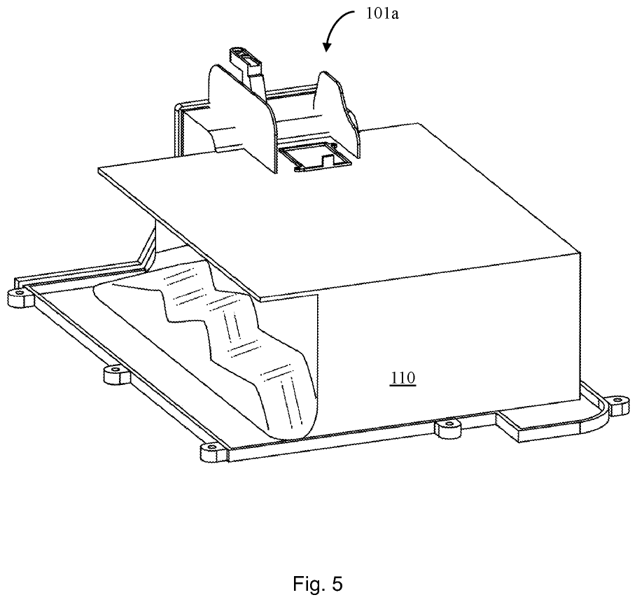

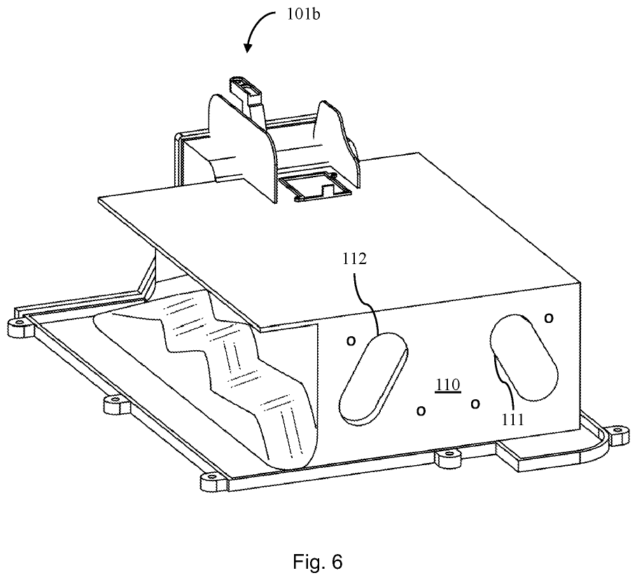

Referring now to FIGS. 5-6, illustrated therein are perspective views of various exemplary “clamshell” enclosures. While drawn to scale, those ordinarily skilled in the art will appreciate that many possible modifications to the same enclosure may achieve similar results to the modifications as disclosed herein. Similarly, those having ordinary skill in the art will appreciate that many clamshell enclosures may exist in different shapes, sizes, designs, and may contain various features, components, parts, etc., which may influence decision-making when implementing the system of the disclosure. The disclosure is not so limited to the specifically contemplated modifications and components modified as illustrated and described herein.

Having full view of the various components of the system of the disclosure as illustrated therein FIG. 4, one can appreciate various features and options to determine the best configuration for any given vehicle. Given that a space at or near airstream enclosure 100 may be very close to other features and equipment, causing a very tight or cramped space there around airstream enclosure 100, electrical components of the system of the disclosure may be housed remotely within the vehicle, and be powered and controlled via a wiring or other remotely controlled and/or powered design. Starting with the inverter, or power converter 220, it may accept 12V DC [VDC] power from the battery of the vehicle, the alternator, or other power sources within the vehicle. By intercepting the wiring providing power from the battery source to, for instance, the HVAC fan, a UVC lighting system consuming approximately 2.5 A of power may be powered. In other potentially preferred embodiments, this may be consuming 2.2 A of power. Connections thereof may be easily facilitated (for ease of installation) via wiring harnesses such as a three-way MOLEX®-type connection (or “quick connection”). Another method may include utilization of two separate connectors which may accommodate two take-offs from each connector. As may be well understood in the art, in order to tap into a circuit, a load wire may accommodate a common wire and potentially a ground wire so as to include the system as herein disclosed within the circuit of an existing vehicle appliance and/or HVAC component (e.g., a fan). Next in series may be ballast 230 which accomplishes two purposes: (a) provides sufficient power at a high enough voltage to “fire” or initiate the lighting of first UVC bulb 301 and second UVC bulb 302 and (b) provides steady state power to keep first UVC bulb 301 and second UVC bulb 302 illuminated during operation. Additional connections may begin/terminate from ballast 230 to first UVC bulb 301 and second UVC bulb 302. The connections at ballast 230 may be of the “push”, harness, or MOLEX® variety and the connections to first UVC bulb 301 and second UVC bulb 302 may first be made into a socket, which in turn fastens to, for instance, the ‘pin’ connections on each of first UVC bulb 301 and second UVC bulb 302. The sockets of each of first UVC bulb 301 and second UVC bulb 302 may additionally serve as the device that mounts that first UVC bulb 301 and second UVC bulb 302 are fastened/secured to airstream enclosure 100. However, bulb sockets and lamp connections may not be sufficiently robust, depending on specific bulb configuration. Therefore, additional modifications, such as those herein described, may be required to ensure sufficient stabilization for first UVC bulb 301 and second UVC bulb 302 during a vehicle's operation. Such modifications may be made to airstream enclosure 100, to first UVC bulb 301 and second UVC bulb 302 themselves, or to the sockets of first UVC bulb 301 and second UVC bulb 302. Additionally, brackets, mounts, gaskets, encapsulating socket(s), and other stabilizing apparatuses which may or may not stabilize at one, two, or more ends of UVC bulbs 301 and 302 may be used. Though described in greater detail above, first UVC bulb 301 and second UVC bulb 302 may be 35 W bulbs, each capable of providing 11 W of UVC light. First UVC bulb 301 and second UVC bulb 302 may be fitted as herein illustrated and described throughout. In various preferred embodiments of the disclosed system, airstream enclosure 100 may cover the cooling coil in the HVAC system, which may allow full airflow from the discharge of the fan, turning the stream of air 90 degrees before entering the coil. Since first UVC bulb 301 and second UVC bulb 302 only project into the interior of the clamshell enclosure, where the air is directed, the forced air through airstream enclosure and past first UVC bulb 301 and second UVC bulb 302, thereby ensuring the stream of air will pass near (i.e., close proximity) to first UVC bulb 301 and second UVC bulb 302, prior to entering the cooling coil of a vehicle HVAC system. Particularly important may be a design which ensures that any airborne organism, (such as a pathogen, virus, etc.) cannot pass through airstream enclosure 100 at a distance greater than 1½ inches from the bulb of either of first UVC bulb 301 and second UVC bulb 302. By causing (i.e., forcing) the stream of air to pass in close proximity to either of first UVC bulb 301 and second UVC bulb 302, a maximum kill rate of the pathogens may be accomplished. As detailed below in a comparison of FIGS. 5-6, modifications to a potentially preferred and/or exemplary clamshell enclosure can accommodate first UVC bulb 301 and second UVC bulb 302.

Referring specifically to FIG. 5, therein illustrated is a perspective view of an unmodified exemplary clamshell version of airstream enclosure 100. It should be recognized by those having skill in the art that, as herein illustrated, only one portion of the clamshell is illustrated. Interestingly, a two-part airstream enclosure 100, such as a clamshell airstream enclosure 100, may only require modification to one of the two parts, thereby increasing the overall value of the proposed modifications to the vehicle HVAC in order to disinfect the air therein. As illustrated, the unmodified version of airstream enclosure 100, unmodified clamshell enclosure 101a, may generally feature a face such as airstream enclosure aperture side 110, though it features no aperture, merely a convenient place to place one or more apertures and thereby install first UVC bulb 301 and second UVC bulb 302.

Referring specifically to FIG. 6, therein illustrated is a perspective view of exemplary modified clamshell enclosure 101b. The modifications to modified clamshell enclosure 101b, in relation to that of 101a, may generally include features which allow first UVC bulb 301 and second UVC bulb 302 to be housed therein. As illustrated herein, two apertures, first airstream enclosure aperture 111 and second airstream aperture 112 have been fashioned and/or molded to be included. Additional features of airstream enclosure aperture side 110 may generally include smaller apertures (i.e., holes) by which screws, bolts, brackets, fasteners, the like and/or combinations thereof may be secured to both first UVC bulb 301 and second UVC bulb 302 as well as airstream enclosure 100 or modified clamshell enclosure 101b so as to stabilize and secure first UVC bulb 301 and second UVC bulb 302 during transit/use.

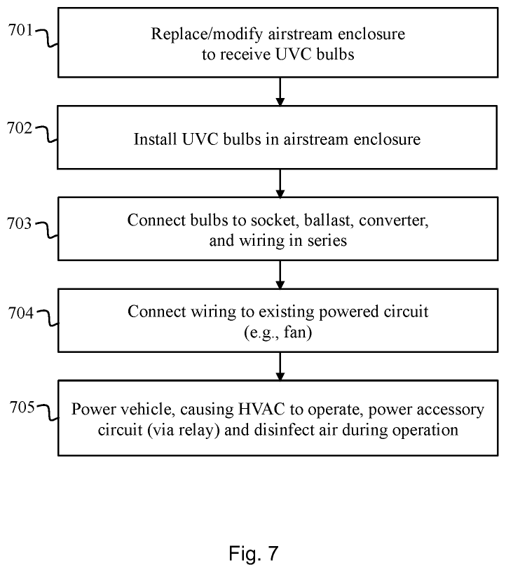

Referring specifically to FIG. 7, shown therein is a flow diagram of an exemplary method of installation. As described herein, few modifications may be required to existing vehicle HVAC systems in order to provide disinfecting power via first UVC bulb 301 and second UVC bulb 302. First, at method step 701, having designed a modified airstream enclosure 100 to accept first UVC bulb 301 and second UVC bulb 302 via a first airstream enclosure aperture 111 and second airstream enclosure aperture 112, respectively, first UVC bulb 301 and second UVC bulb 302 may be installed in and secured to airstream enclosure 100 at method step 702. In order to power first UVC bulb 301 and second UVC bulb 302, connections may be made to ballast 230, power converter 220, then from power converter 220 to a power source via power source wiring 221 at method step 703. Since many components within a vehicle may already receive power, one such circuit may be utilized to provide power to first UVC bulb 301 and second UVC bulb 302 at method step 704. In potentially preferred embodiments, power is relayed via permissive relay R so first UVC bulb 301 and second UVC bulb 302 may receive power from circuit C, which may receive power from an accessory circuit, such as a fan, main vehicle power, or other vehicle circuit. During normal operation of vehicle HVAC system, first UVC bulb 301 and second UVC bulb 302 may receive power and illuminate airstream enclosure 100 as described herein.

The illustrations and diagrams (hereafter “illustrations”) described herein are intended to provide a general understanding of the structure of various embodiments of the disclosed system and method of vehicle airstream disinfection. The illustrations are not intended to serve as a complete description of all of the elements and features of the apparatus, product, method of use, and/or system that utilizes the structures and/or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

With respect to the above description then, it is to be realized that the optimum dimensional relationships, to include variations in size, materials, shape, form, position, function and manner of operation, assembly, type of airflow mechanism, power supply, type of bulb(s), shape of housing(s), material(s) of housing, mounting location(s), vehicle type and use, all of which are intended to be encompassed by the present disclosure.

It is contemplated herein that the device may include a variety of overall sizes and corresponding sizes for and of various parts, including but not limited to: UVC lights, transformer(s), converter(s), ballast(s), interconnecting wiring, relay(s), switch(es), electrical connections (e.g., MOLEX®), electrical enclosures, and redesigned “clamshell” enclosure(s). Furthermore, it is contemplated that due to variations in vehicles, their size and shape, their onboard power systems, their ventilation and/or HVAC systems, including but not limited to tractor trailers and semi-trucks, passenger cars, passenger trucks, cable cars, buses, ferries, ships, boats, train cars, locomotives, submersibles, aircraft, the like and/or any combination of enclosed transport thereof, adjustments may be made to the disclosed device to better adapt the system of the disclosure to the intended enclosed transport vehicle. Yet still, though the inventor has contemplated one method of sterilizing the air contained in enclosed transport vehicles, the disclosure is not limited to specific UVC lights, transformer(s), converter(s) ballast(s), interconnecting wiring, relay(s), switch(es), electrical connections (e.g., MOLEX®), electrical enclosures, and redesigned “clamshell” enclosure(s). Other means that have been contemplated and which the disclosed system and method may include, but the disclosure is not so limited, variations in air sterilization methods (e.g. other UV wavelengths, heat, steam, pressure, ionization), variations in amperage and voltage of transformers, variation in size/shape/material for ballast, variation in wiring and corresponding connections/harnesses, as well as size, shape, and material for construction of an enclosure. Various trade-offs may be considered when selecting the technology to deploy in the system and method for UVC airstream disinfection. These include limiting the power consumption during use or idle, installing an on/off switch, including new methods of air sterilization as they are developed and/or discovered, including filtration devices within the system to collect airborne particles subsequent and/or prior to UV treatment, sensors and/or indicators to determine operability, air quality, light or UVC radiation intensity during operation, and level of operation, and/or status lights and/or interconnectivity of the device to third party devices via a variety of technologies including but not limited to: onboard vehicle systems, onboard vehicle diagnostic systems/computers (e.g. OBD), wired or wireless computing devices that may connect to such systems or to the device itself, cloud systems that may remotely connect and monitor the device through long-range wireless technologies (e.g. 3G or LTE). It is also contemplated that certain considerations and/or additional features of the present disclosure may improve the functionality. These may include communication protocols to alert of bulb health, filter life and airflow quality, cleaning and sanitization protocols, and the like and combinations thereof. In regard to communication with other devices via a network, the device may communicate via any known or yet to be discovered protocol, including wired networking, fiber optic communication, wireless networking (i.e. WiFi), near field communication (e.g. Bluetooth® or NFC), the like or combinations thereof. The device may receive power from the vehicle in which it is designed to be installed, a separate power source external and/or temporary to the vehicle (e.g., when a driver exits a vehicle and a new driver enters), a solar and/or wind energy source installed onto the vehicle, or may contain a battery which may or may not have the capability to re-charge.

The foregoing description and drawings comprise illustrative embodiments of the present disclosure. Having thus described exemplary embodiments, it should be noted by those ordinarily skilled in the art that the within disclosures are exemplary only, and that various other alternatives, adaptations, and modifications may be made within the scope of the present disclosure. Merely listing or numbering the steps of a method in a certain order does not constitute any limitation on the order of the steps of that method. Many modifications and other embodiments of the disclosure will come to mind to one ordinarily skilled in the art to which this disclosure pertains having the benefit of the teachings presented in the foregoing descriptions and the associated drawings. Although specific terms may be employed herein, they are used in a generic and descriptive sense only and not for purposes of limitation. Moreover, the present disclosure has been described in detail, it should be understood that various changes, substitutions and alterations can be made thereto without departing from the spirit and scope of the disclosure as defined by the appended claims. Accordingly, the present disclosure is not limited to the specific embodiments illustrated herein, but is limited only by the following claims.

What is claimed is:

1. A UVC airstream disinfection system for vehicles comprising:
a first module, said first module is configured to receive a vehicle cabin air intake and an at least one vehicle cabin air outlet, said first module further comprising an at least one aperture, the at least one aperture is capable of receiving an at least one UVC bulb; and
a power supply capable of continuously powering said at least one UVC bulb and operably connected thereto;
wherein as an airstream passes through said first module, said airstream is continuously supplied with UVC radiation via said at least one UVC bulb.

2. The system of claim 2, wherein said power supply comprises a ballast, a converter, and a connection to a vehicle accessory circuit.

3. The system of claim 3, wherein said ballast and said converter are housed within a second module, said second module is an enclosure.

4. The system of claim 3, wherein said ballast features a normal input voltage of 24 VDC, an input current of 2.5 A(24V), an input current total harmonic distortion of less than 10%, an operating frequency of >35 kHz, and a lamp current crest factor of 1.7 or less.

5. The system of claim 3, wherein said converter converts a 12V direct current to 24 VDC current capable of powering said at least one UVC bulb.

6. The system of claim 1, wherein said at least one UVC bulb is a pair of UVC bulbs.

7. The system of claim 6, wherein each of said pair of UVC bulbs comprises a cap-base 2G11 having 4 pins, a color code TUV (UVC), a 0.85 A current, a 225 mm length, a 35 W power rating, a 254 nm spectrum, a 11 W total output, and an expected 9,000-hour useful life.

8. The system of claim 3, wherein a plurality of electronic connections are installed therebetween each of said pair of UVC bulbs and said ballast, and said converter and said connection to said vehicle accessory circuit.

9. The system of claim 8, wherein said vehicle accessory circuit is a vehicle fan circuit.

10. The system of claim 9, wherein said airstream, while traversing within said first module, does not exceed 1.5″ from said at least one UVC bulb.

11. The system of claim 1, wherein said first module is an enclosure of a vehicle HVAC system, said enclosure further comprises a mounting bracket capable of securing said UVC bulb to said enclosure.

12. A method of continuously disinfecting an air of a vehicle cabin, the method comprising:
installing a first module at a vehicle HVAC manifold having at least an air outlet and an air inlet, said first module comprising:
an aperture capable of receiving an at least one UVC bulb; and
said at least one UVC bulb;
powering said first module via wiring from a second module connected to a vehicle accessory circuit; and
operating the vehicle HVAC system, thereby disinfecting the air through a continuous supply of a UVC radiation from said at least one UVC bulb.

13. The method of claim 12, further comprising a step of modifying said vehicle HVAC manifold to include said aperture.

14. The method of claim 13, wherein said at least one UVC bulb is a pair of UVC bulbs.

15. The method of claim 14, wherein each of said pair of UVC bulbs comprises a cap-base 2G11 having 4 pins, a color code TUV (UVC), a 0.85 A current, a 225 mm length, a 35 W power rating, a 254 nm spectrum, a 11 W total output, and an expected 9,000-hour useful life.

16. The method of claim 15, wherein said second module further comprises a ballast and a transformer.

17. The method of claim 16, wherein said vehicle accessory circuit is a vehicle fan circuit.

18. The method of claim 17, wherein during operation of a vehicle fan, the air, while traversing within said first module, does not exceed 1.5″ from said at least one UVC bulb.

19. The method of claim 18, wherein said first module further comprises a mounting bracket capable of securing said pair of UVC bulbs.

20. The method of claim 16, wherein said converter converts a 12V direct current to 24 VDC current capable of powering said at least one UVC bulb.
